(19) United States
(12) Patent Application Publication
(10) Pub. No.: US 2006/0115873 A1
(43) Pub. Date: Jun. 1, 2006

(54) Novel exothermic material, method and device for lysing cells using the exothermic material

(76) Inventors: Sang-hyun Peak, Seoul (KR); Jung-nam Lee, Incheon-si (KP); Jong-myeon Park, Seoul (KR); Jong-Suk Chung, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/289,857

(22) Filed: Nov. 30, 2005

(30) Foreign Application Priority Data
Dec. 1, 2004 (KR) 10-2004-0099743

(51) Int. Cl. C12P 1/00

(52) U.S. Cl. 435/41

(57) ABSTRACT

Provided are an exothermic material, and a method and device for lysing cells using the exothermic material. The exothermic material is a solid material containing CaO, 5-7% by weight of MgCl₂ or MgSO₄, and 5-7% by weight of NaOH, with respect to the total weight of CaO, and generates heat upon the addition of sulfuric acid.

### CROSS-REFERENCE TO RELATED PATENT APPLICATION

This application claims the benefit of Korean Patent Application No. 10-2004-0099743, filed on Dec. 1, 2004, in the Korean Intellectual Property Office, the disclosure of which is incorporated herein in its entirety by reference.

### BACKGROUND OF THE INVENTION

1. Field of the Invention

The present invention relates to a novel exothermic material, and a method and device for lysing cells using the same.

2. Description of the Related Art

In the conventional art, there are known compositions which generate heat as a result of electrochemical reactions. For example, Korean Laid-open Patent Application No.1990-004891 discloses a composition generating heat as a result of the reaction between quicklime (CaO) and a solution of MgCl₂. Also known is that heat is generated when quicklime (CaO) is reacted with a strong acid (for example, HCl and H₂SO₄). Korean Laid-open Patent Application No. 2000-58524 discloses a method of heating food by an exothermic hydration reaction in which quicklime is reacted with phosphorus or a phosphate compound. Further, Korean Laid-open Patent Application No. 2001-35352 discloses an exothermic material which generates heat when contacting a solid substance including CaO, AlCl₃, NaOH and metallic Al, with NaCl, CaCl₂, MgCl₂, NH₄Cl or Fe(NO₃)₃, each of which are in the liquid phase.

However, these conventional technologies relates to exothermic materials that have been derived for the purpose of heating foodstuffs, and use of these materials for lysing cells has not been reported yet. The conventional exothermic materials are unsatisfactory for rapidly providing a large quantity of heat for cytolysis.

Therefore, as it is obvious from the prior art as described, there still is a demand for an exothermic material which generates a sufficiently large quantity of heat rapidly so as to be used in cytolysis. The inventors of the present invention have carried out research on such exothermic materials capable of rapidly generating a large quantity of heat and have discovered a subtitled exothermic material.

### SUMMARY OF THE INVENTION

The present invention provides an exothermic material which can generate a large quantity of heat rapidly.

The present invention provides a method of lysing cells using the above-described exothermic material.

The present invention provides a device for cytolysis containing the above-described exothermic material. According to an aspect of the present invention, there is provided an exothermic material which is a solid material containing CaO, 5-7% by weight of MgCl₂ or MgSO₄ and 5-7% by weight of NaOH, with respect to the total weight of CaO, and generates heat upon the addition of sulfuric acid.

According to another aspect of the present invention, there is provided a method of lysing cells using heat generated by an electrochemical reaction, the method comprising: introducing cells into a container; and transferring to the container the heat generated upon the addition of a 0.1 to 5 M sulfuric acid solution to a solid material containing CaO, 5-7% by weight of MgCl₂ or MgSO₄, and 5-7% by weight of NaOH, with respect to the total weight of CaO.

According to another aspect of the present invention, there is provided a device for lysing cells using heat generated by an electrochemical reaction, the device comprising: a container to hold cells; and a compartment adjacent to the container, the compartment containing a solid exothermic material containing CaO, 5-7% by weight of MgCl₂ or MgSO₄, and 5-7% by weight of NaOH, with respect to the total weight of CaO, and having an inlet for introducing sulfuric acid.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a device according to an embodiment of the present invention in which a compartment for exothermic reaction is formed by assembling a first container and a second container;

FIG. 2 is a photograph showing the result of electrophoresis of a polymerase chain reaction (PCR) product obtained by lysing cells according to the method of v invention and conducting PCR using the supernatant obtained through the centrifugation of the lysate as a template;

FIG. 3 is a graph showing the measurement results of the amounts of PCR products in some lanes in FIG. 2 using a Labchip (available from Agilent Technologies);

FIG. 4 is a diagram illustrating a device according to another embodiment of the present invention in which a container for cytolysis 10 and a compartment 20 for exothermic reaction formed with a cell inlet 24 and containing an exothermic material 22 are integrated; and

FIG. 5 is a diagram illustrating a device according to another embodiment of the present invention in which a container for cytolysis 10 and a compartment 20 for exothermic reaction containing an exothermic material 22 are formed as microchannels.

### DETAlLED DESCRIPTION OF THE INVENTION

The present invention provides an exothermic material generating heat upon the addition of sulfuric acid that is a solid material containing CaO, 5-7% by weight of MgCl₂ or MgSO₄ and 5-7% by weight of NaOH, with respect to the total weight of CaO.

The exothermic material according to the present invention, which contains CaO, 5-7% by weight of MgCl₂ or MgSO₄ and 5-7% by weight of NaOH, with respect to the total weight of CaO, is prepared by these compounds in any order to yield a solid-state material. According to the invention, this solid-state material can be in any arbitrary form, such as powder, plate, lump, etc. The solid-state material in such a form can be molded into an appropriate shape in accordance with the purpose for which the exothermic material is to be used and the place where it is to be used.

The exothermic material according to the invention can generate a large amount of heat in a very short time when it contacts sulfuric acid. For example, in a reaction with sulfuric acid for about 10 seconds, the material can be raised to a temperature of about 130° C. The exothermic material according to the present invention is designed to be able to generate a larger amount of heat in a short time as a result of combined exothermic reactions that are already known in the art. That is to say, a solid material containing all of CaO, MgCl₂ and NaOH, which are reactants used in exothermic reactions with sulfuric acid, is prepared, and then an exothermic reaction is induced by adding sulfuric acid thereto. The obtained exothermic material according to the present invention invention can provide a larger amount of heat than the heat generated through the reaction between CaO and sulfuric acid and the reaction between MgCl₂, NaOH, and sulfuric acid. The heat generated by the exothermic material according to the present invention can be used in various applications. For example, it can be used in a method for lysing cells using heat.

The present invention also provides a method of lysing cells using the heat generated in an electrochemical reaction, the method including: introducing cells into a container; and transferring to the container the heat generated upon the addition of a 0.1 to 5 M sulfuric acid solution to a solid material containing CaO, 5-7% by weight of MgCl₂ or MgSO₄ and 5-7% by weight of NaOH, with respect to the total weight of CaO.

According to the present invention, the container can be anything having a space in which cells or a solution containing cells can be placed. For example, the container may be a microchannel or a cavity in a microfluidic device. Here, the microfluidic device refers to a device comprising a solid support having microchannels through which a fluid can flow into inlets, outlets, wells, a reaction chamber, and the like. In such a microfluidic device, an element for driving the fluidic flow, such as a pump, is used to move the fluid. The microfluidic device is disclosed in, for example, EP 0 637 996 B1 and EP 0 637 998 B, the disclosures of which are incorporated in the present specification by reference.

In the method according to the present invention, the cells are introduced into the above-described container. The introducing the cells into the container may include introducing previously cultured cells into the container through an injecting device, or culturing the cells or a sample containing cells in the aforementioned container. Next, the heat generated upon the addition of a 0.1 to 5 M sulfuric acid solution to a solid material containing CaO, 5-7% by weight of MgCl₂ or MgSO₄ and 5-7% by weight of NaOH, with respect to the total weight of CaO, is transferred to the container to lyse the cells in the container by the heat. In the transferring the heat generated through the exothermic reaction to the container, the heat can be transferred by direct conduction while it is generated through the reaction between the exothermic material, which is brought to contact the container, and sulfuric acid, or can be transferred by convection and radiation, but the route of the heat transfer is not limited to these examples. During the heat transfer, the cell solution in the container may be stirred to allow easy heat transfer.

The present invention also provides a device for lysing cells by the heat generated by an electrochemical reaction, the device includes a container to hold cells and a compartment adjacent to the container, wherein the compartment contains a solid material containing CaO, 5-7% by weight of MgCl₂ or MgSO₄ and 5-7% by weight of NaOH, with respect to the total weight of CaO, and has an inlet through which sulfuric acid is injected.

In the present invention, the container and the compartment can be microchannels or microchambers. An example of the device according to the present invention is a microfluidic device having a microchannel or microchamber which holds cells, and another adjacent microchannel or microchamber which contains a solid exothermic material containing CaO, 5-7% by weight of MgCl₂ or MgSO₄ and 5-7% by weight of NaOH, with respect to the total weight of CaO. Thus, the device for lysing cells according to an embodiment of the present invention may have a region for receiving cell lysate from the cell container and separating a substance, such as nucleic acid, from the cell lysate. Alternatively, the device according to the present invention may further comprise a PCR unit in which PCR is performed on the cell lysate or the nucleic acid separated therefrom.

In the device according to the present invention, the exothermic material may be contained in any arbitrary form in the compartment. For example, the exothermic material may be contained in the form of a coating layer on the inner wall of the compartment. The compartment may be built in the device or may form a separate unit, which is later assembled with the cell container.

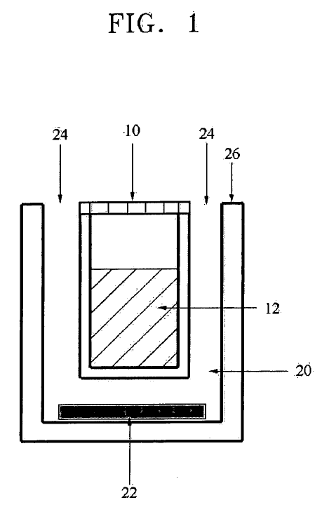

The device of the invention can be implemented in various forms or shapes. FIG. 1 illustrates a device for lysing cells by heat according to an embodiment of the present invention in which the compartment for exothermic reaction is formed by assembling a first container 10 and a second container 26. In FIG. 1, the first container 10, which can hold cells 13, is placed in the second container 26, forming a compartment 20 for exothermic reaction occurs, the compartment 20 having an inlet 24 through which sulfuric acid is introduced. The first container 10 and the second container 26 can be separated from one another. The bottom of the second container 26 is coated with an exothermic material 22 according to the present invention.

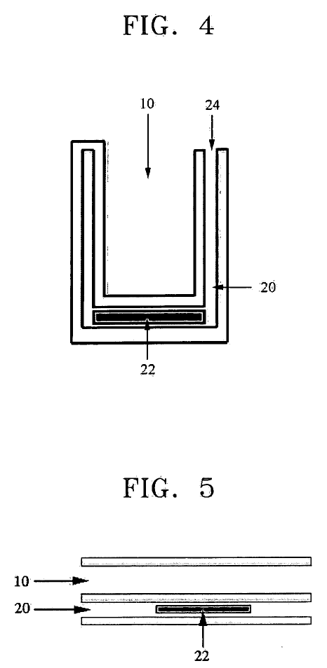

FIG. 4 is a diagram illustrating a device according to another embodiment of the present invention. In the device of FIG. 4, a container 10 for cytolysis and a compartment 20 where an exothermic reaction takes place, the compartment 20 having an inlet 24 through which cells are injected and containing the exothermic material 22, are integrally formed.

FIG. 5 is a diagram illustrating a device according to another embodiment of the present invention. In the device of FIG. 5, the container 10 for cytolysis and the compartment 20 containing the exothermic material 22 and in which an exothermic reaction takes place are formed as microchannels.

Hereinafter, the present invention will be described in more detail with reference to the following examples. The following examples are for illustrative purposes and are not intended to limit the scope of the present invention.

### EXAMPLE 1

### Comparison of the Amounts of Heat Generated through Conventional Exothermic Reactions

In the current example, a comparison was made on the quantities of heat generated through conventionally known reactions between each of MgSO₄, NaOH and CaO, and water.

The device for cytolysis shown in FIG. 1 was used. First, 50 μL of Escherichia coli (E. coli) cells (OD₆₀₀=1) 12 were introduced into a glass tube 10 having a volume of 2 mL, which was then capped and sealed. Next, a coating layer was formed by applying 2 g of each of ( [symbol] ) MgSO₄, NaOH, and CaO to a glass bottom of the second container 26, and 3 mL of water was introduced through the inlet 24 into the compartment 20, which was defined by the second container 26 and the glass tube 10. Then, a reaction between each of the layers and water was allowed for 5 minutes. The temperature of each of reaction solutions in the compartment 20 was measured at the inlet during the reaction. As a result, the temperature was 80° C. for the MgSO₄, 75° C. for the NaOH, and 70° C. for the CaO layer, which are shown in Table 1. However, these temperature levels were considered to be insufficient for lysing cells.

TABLE 1

| Reaction | Temperature |
|---|---|
| MgSO₄ + H₂O | 80° C. |
| NaOH + H₂O | 75° C. |
| CaO + H₂O | 70° C. |

Among the three exothermic reactions described above, the reaction involving CaO, which allows both hydration and neutralization reactions was selected to develop an exothermic material generating a larger amount of heat.

### EXAMPLE 2

### Confirmation of the Amount of Heat Generated by Exothermic Material According to the Present Invention

Using the device for cytolysis illustrated in FIG. 1, the amount of heat generated through the reaction between CaO and sulfuric acid, the amount of heat generated through the reaction between MgCl₂, NaOH and sulfuric acid, and the amount of heat generated through the reaction between CaO, MgCl₂, NaOH and sulfuric acid were measured.

First, 50 μL of E. coli cells (OD₆₀₀=1) 12 was introduced into the glass tube 10 having a volume of2 ml, which was then capped and sealed. Next, 2 g of CaO, 2 g of a mixture of MgCl₂ and NaOH (100% by weight of NaOH with respect to 100% by weight of MgCl₂), and 2 g of a mixture of CaO, MgCl₂, and NaOH (7% by weight of MgCl₂ and 7% by weight of NaOH, with respect to 100% by weight of CaO), were respectively applied to the glass bottoms of the second containers 26 in the compartments 20 to form coating layers. Then, 3 mL of 1 M sulfuric acid was introduced into the inlet 24 into each of the compartments 20, which were defined by the second container 26 and the glass tube 10, and reacted. At certain times, the temperature of each of cell solutions 12 was measured. As a result, the exothermic material according to the invention, which contained 2 g of a mixture of CaO, MgCl₂ and NaOH (7% by weight of MgCl₂ and 7% by weight of NaOH, with respect to the total weight of CaO) generated the largest amount of heat among the three different exothermic materials, which is shown in Table 2.

TABLE 2

| | Temperature at different | | |
|---|---|---|---|
| | reaction times (sec) | | |
| Reaction | 10 sec | 60 sec | 90 sec |
| CaO + H₂SO₄ | 100° C. | 85° C. | 78° C. |
| MgCl₂ + NaOH + H₂SO₄ | 110° C. | 92° C. | 73° C. |
| CaO + MgCl₂ + NaOH + H₂SO₄ | 130° C. | 100° C. | 95° C. |

### EXAMPLE 3

### Cytolysis by the Exothermic Reaction Using the Exothermic Material According to the Invention

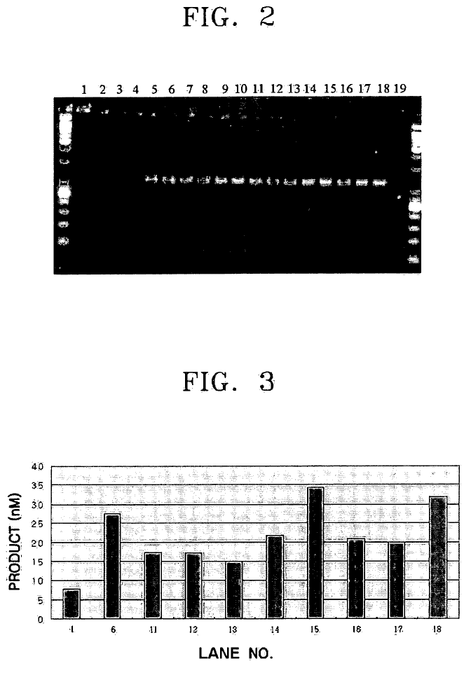

In the current example, cells were lysed using the exothermic material of Example 2 consisting of CaO, MgCl₂ and NaOH (7% by weight of MgCl₂ and 7% by weight of NaOH, with respect to the total weight of CaO), and the supernatant of the lysate was subjected to spin-down centrifugation to remove solid components. 1 μL of an aliquot was taken from the liquid layer, and PCR was performed using the aliquot as a template and oligonucleotides of SEQ ID Nos. 1 and 2 as primers. The resulting PCR product was subjected to electrophoresis in polyacrylamide gel. FIG. 2 illustrates the electrophoresis results for the PCR product obtained when cells were lysed according to the method according to the present invention and PCR was performed using the supernatant obtained by the centrifugation of the lysate as a template. In FIG. 2, lane 1 corresponds to positive control (a cell solution was centrifuged, without lysing cells, to remove the solid component, a 1-μL aliquot of the supernatant was taken from the liquid layer, and PCR was performed using the aliquot as a template); and lanes 2 through 6 correspond to samples exposed to ultrasonic waves for 15 sec, 45 sec, 1 min, and 2 min, respectively; lanes 7 through 13 correspond to samples in which cells were lysed by boiling for 15 sec, 30 sec, 1 min, 2 min, 3 min, 4 min and 5 min, respectively; lanes 14 through 18 correspond to samples in which cells were lysed according to the method according to the present invention which was repeated 5 times; and lane 19 corresponds to negative control. The cytolysis using ultrasonic waves was carried out using a home microwave oven set at a high level of 2.45 Ghertz for 15 sec to 2 min. The cytolysis by boiling was carried out at 95° C. for 15 sec to 5 min.

FIG. 3 is a graph illustrating the results of measuring the amounts of PCR products in some lanes in FIG. 2 using a Labchip (available from Agilent Technologies). As shown in FIGS. 2 and 3, when the method of lysing cells using the exothermic reaction according to the present invention is used, cells can be lysed with at a higher efficiency than when the conventional ultrasonic method and the boiling method are used.

The exothermic material according to the present invention can generate a large quantity of heat rapidly.

The method of lysing cells according to the present invention allows rapid cytolysis.

The device for lysing cells according to the present invention can be used to lyse cells rapidly and is portable since it does not require an electric power source.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An exothermic material which is a solid material containing CaO, 5-7% by weight of MgCl₂ or MgSO₄ and 5-7% by weight of NaOH, with respect to the total weight of CaO, and generates heat upon the addition of sulfuric acid.

2. A method of lysing cells using heat generated by an electrochemical reaction, the method comprising:
introducing cells into a container; and
transferring to the container the heat generated upon the addition of a 0.1 to 5 M sulfuric acid solution to a solid material containing CaO, 5-7% by weight of MgCl₂ or MgSO₄, and 5-7% by weight of NaOH, with respect to the total weight of CaO.

3. The method according to claim 2, wherein the container is a microchannel or a cavity in a microfluidic device.

4. A device for lysing cells using heat generated by an electrochemical reaction, the device comprising:
a container to hold cells; and
a compartment adjacent to the container, the compartment containing a solid exothermic material containing CaO, 5-7% by weight of MgCl₂ or MgSO₄, and 5-7% by weight of NaOH, with respect to the total weight of CaO, and having an inlet for introducing sulfuric acid.

5. The device according to claim 5, wherein the container and the compartment are microchannels or microchambers.


SEQUENCE LISTING (2 sequences)

SEQ ID NO: 1 - DNA, 20 bp, Artificial
  primer
ccakactcctacgggaggcw

SEQ ID NO: 2 - DNA, 22 bp, Artificial
  primer
gtgccagcagyygcggtaatac